(19) United States
(12) Patent Application Publication
(10) Pub. No.: US 2012/0282308 A1
(43) Pub. Date: Nov. 8, 2012

(54) FILM CONTAINING ALGINATE MEMBRANE AND MANUFACTURING METHOD OF THE SAME

(76) Inventors: Yao-Chung CHIU, Keelung City (TW); Yu-Yueh Lin, Taipei City (TW)

(21) Appl. No.: 13/240,401

(22) Filed: Sep. 22, 2011

(30) Foreign Application Priority Data
May 4, 2011 (TW) 100115636

(51) Int. Cl. A61K 8/02; B05D 3/10

(52) U.S. Cl. 424/401; 427/339; 427/2.31

(57) ABSTRACT

A film containing alginate membrane and a manufacturing method of the same are revealed. A film carrier is used to absorb liquid and is treated by two separate and continuous coating processes. At least one surface of the film carrier is coated with a layer of alginate solution containing a certain percent of sodium alginate or potassium alginate by weight and then is coated with a layer of salt water solution containing a certain percent of divalent metal ions. Then crosslinking occurs between the alginate in the alginate solution and the divalent metal ions in the salt solution on the surface of the film carrier or infiltrating into the film carrier so as to form a hydrogel complex membrane having a network structure. Thus the continuous and fast mass-production of the film is achieved.

### BACKGROUND OF THE INVENTION

The present invention relates to a film containing at least one alginate membrane and a manufacturing method of the same. The film includes a film carrier and an alginate membrane. The film carrier absorbs liquid. At least a layer of alginate solution and a layer of slat water solution containing divalent metal ions are coated on a surface of the film carrier by two separate and continuous processes. Then crosslinking occurs between the two solutions on the surface of the film carrier to produce the film containing an alginate membrane.

Alginic acid is a natural copolymer, easily reacted with metal ions from salts to form various kinds of alginate for commercial uses. The water-soluble sodium alginate reacts with double charged calcium ion (Ca²⁺) from salts so as to form insoluble calcium alginate gel. Alginate products for commercial use include sodium alginate, potassium alginate, ammonium alginate, etc, being applied to wound dressings or facial masks. There are several methods for making alginate gels available now, including:

(1) dialysis/diffusion method: this is the most commonly used method. The alginate solution is gelled by diffusion of calcium ions from an outer reservoir.

(2) in situ gelling: a calcium salt with limited solubility is mixed with a sodium alginate solution and then a slowly acting acid is added into the mixture. Due to the acid, the calcium ions are released to cross-link the alginate molecules so as to form calcium alginate gel.

(3) cooling method: at high temperature, calcium ions are unable to bond with alginic acid. Thus dissolve sodium alginate and calcium salts in hot solution. Then allow the solution to set and form gel by cooling.

(4) crosslinking method: the hydrogen groups contained in alginate molecules are cross-linked by epichlorohydrin (ECH) so as to form gel insoluble in solution.

Now the alginate or similar materials have been applied to wound dressings or facial masks, as the alginate revealed in U.S. Pat. No. 6,080,420, U.S. Pat. No. 6,258,995, U.S. Pat. No. 6,203,845, U.S. Pat. No. 6,201,164, U.S. Pat. No. 6,372,248, U.S. Pat. No. 6,326,524, U.S. Pat. No. 5,144,016, U.S. Pat. No. 5,230,853, U.S. Pat. No. 5,622,666, U.S. Pat. No. 5,660,857, U.S. Pat. No. 5,675,957, PCT/GB 9502284 (WO96/10106), PCT/GB 9601719 (WO97/03710), PCT/GB 9701098 (WO97/39781), PCT/DK 9700292 (WO98/02196), TW 95218502, TW201100119A1, TW I265814, etc. However, most of these prior arts focus on components of the materials or percent by weight of the components. The problems occur or practical requirements during manufacturing of the alginate membrane have not been mentioned, especially how to mass produce the alginate membrane or how to reduce manufacturing cost of the alginate membrane.

Thus there is a need to provide a film containing an alginate membrane and a manufacturing method of the same related to water-soluble sodium alginate or potassium alginate for improving the performance and applications of the film. The manufacturers in this industry have more options.

### SUMMARY OF THE INVENTION

Therefore it is a primary object of the present invention to provide a film containing at least one alginate membrane and a manufacturing method of the same. The film includes a film carrier and at least one alginate membrane. The film carrier can be non-woven fabric (non-woven cloth), textile, a porous sponge film, or a plastic film. The film carrier is made from synthetic fibers, natural fibers, one kind of high molecular polymers or their combinations. The synthetic fibers include polyethylene terephthalate (PET), nylon, acrylics, polypropylene (PP), poly lactic acid (PLA), etc. The natural fibers include cotton, linen, wool, silk, etc. The alginate membrane is a hydrogel complex membrane having a network structure, formed by following steps: provide an alginate solution containing a certain weight of sodium alginate or potassium alginate (in weight percent, wt %) and a salt water solution containing a certain weight of divalent metal ions such as calcium ions. The salt can be calcium lactate, calcium chloride, calcium gluconate, poly-glutamic acid calcium, calcium carbonate, calcium sulfate, etc. Then at least one surface of the film carrier is coated with a layer of the alginate solution and a layer of the salt water solution by at least two separate and continuous processes. There is no restriction on the order of the coating solution. Then crosslinking occurs between alginate and the divalent metal ions in the salt water solution, on the surface of the film carrier or infiltrating into the film carrier to form a hydrogel complex membrane having a network structure. Thus a film containing an alginate membrane is mass-produced continuously and quickly. Therefore, the production cost is reduced due to mass-production of the alginate membrane.

It is another object of the present invention to provide a film containing at least one alginate membrane and a manufacturing method of the same. When the surface of the film carrier (such as non-woven fabric (cloth), textile porous sponge film, etc.) is a rough surface, the alginate membrane formed is connected to the film carrier tightly, not separated easily. When the surface of the film carrier (such as plastic film) is a smooth surface, the alginate membrane formed is able to be separated from the film carrier and used for following processes.

It is a further object of the present invention to provide a film containing at least one alginate membrane and a manufacturing method of the same. After the crosslinking reaction being completed, one or two surfaces of the film carrier is/are spray-coated continuously with a layer of alginate solution and a layer of salt water solution in sequence. Thus the thickness of the alginate membrane formed is easy to be increased and controlled by at least one further crosslinking reaction.

### BRIEF DESCRIPTION OF THE DRAWINGS

FIG. 1 is a schematic drawing showing a cross section of an embodiment having an alginate membrane disposed on a surface of a film carrier according to the present invention;

FIG. 2 is a schematic drawing showing a cross section of an embodiment having an alginate membrane disposed on a surface of a film carrier according to the present invention;

FIG. 3 is a schematic drawing showing arrangement of coating equipments during manufacturing processes of the present invention;

FIG. 4 is a flow chart showing manufacturing processes of a film containing alginate membrane according to the present invention;

FIG. 5 is a flow chart showing manufacturing processes of an embodiment of a film containing alginate membrane according to the present invention;

FIG. 6 is a flow chart showing manufacturing processes of another embodiment of a film containing alginate membrane according to the present invention.

### DETAILED DESCRIPTION OF THE PREFERRED EMBODIMENT

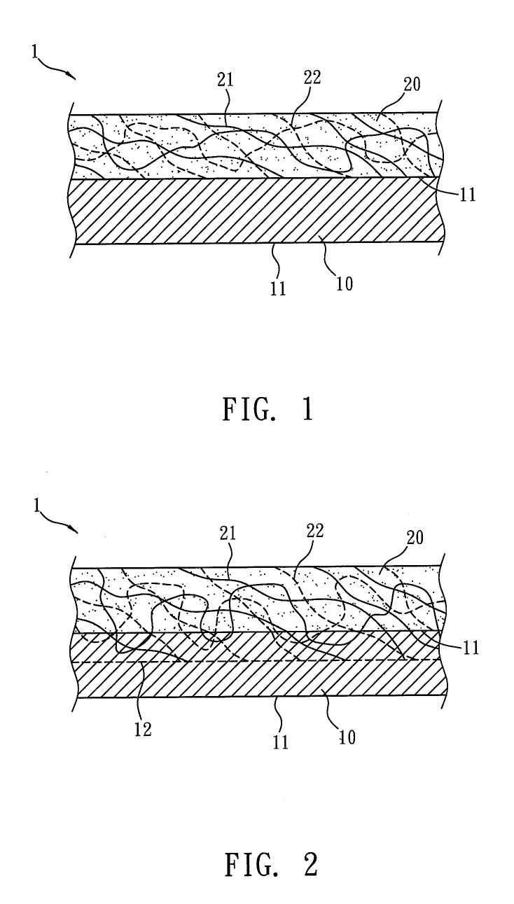

Refer to FIG. 1 and FIG. 2, a film 1 containing at least one alginate membrane according to the present invention includes a film carrier 10, and at least one alginate membrane 20. The film carrier 10 is a carrier that absorbs liquid. The film carrier 10 can be a plastic film with a smooth surface or a thin layer with a rough surface such as non-woven fabric (cloth), fabric or porous sponge. Moreover, the film carrier 10 is made from synthetic fibers, natural fibers, one kind of high molecular polymers or combinations of at least two kinds of high molecular polymers. The synthetic fibers include but not limited to polyethylene terephthalate (PET), nylon, acrylics, polypropylene (PP), poly lactic acid (PLA), etc. The natural fibers include but not limited to cotton, linen, wool, silk, etc. A surface 11 of the film carrier 10 can be a rough surface or a smooth surface according to users' needs or applications.

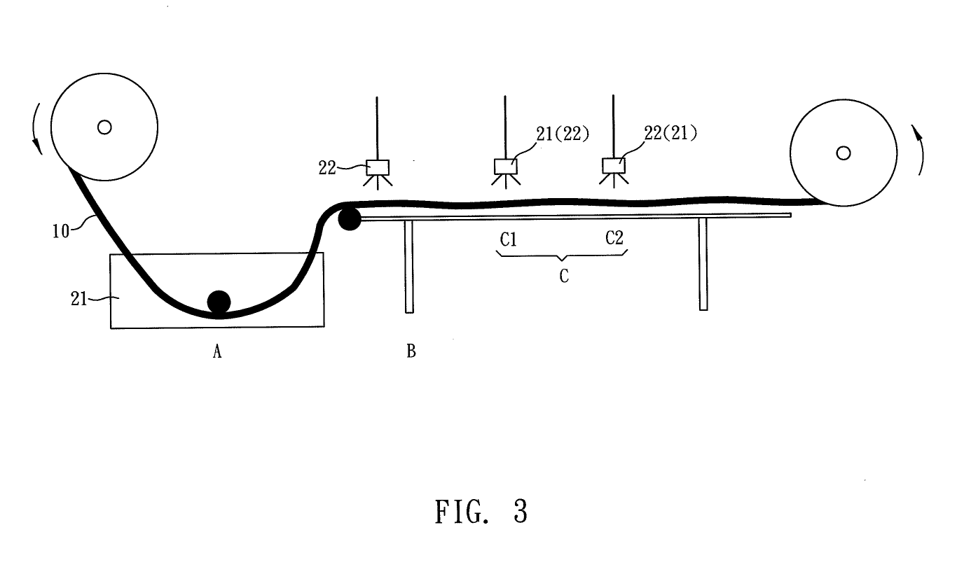

The alginate membrane 20 is a hydrogel complex membrane having a network structure. Refer to FIG. 3, the alginate membrane 20 is produced by following steps:

providing an alginate solution 21 containing a certain weight percent (wt %) of sodium alginate or potassium alginate and a salt water solution 22 containing a certain weight percent (wt %) of divalent metal ions such as calcium ions;

coating a layer of the alginate solution 21 and a layer of the salt water solution 22 evenly on at least one surface 11 of a film carrier 10 by two separate processes carried out successively;

crosslink alginate in the alginate solution 21 with divalent metal ions in the salt water solution 22 on the surface 11 of the film carrier 10 so as to form a hydrogel complex membrane (20) with a network structure on the surface 11 of the film carrier 10 (as shown in FIG. 1), or filtrating into the surface 11 of the film carrier 10 (as shown in FIG. 2). As shown in FIG. 1 and FIG. 2, the alginate membrane 20 is formed by alginate (represented by solid lines) crosslinked with divalent metal ions (represented by dotted lines) to have a crosslinked network structure.

In the present invention, the surface 20 of the film carrier 10 is used to load the alginate solution 21 and the salt water solution 22 so that an alginate membrane 20 such as a calcium alginate membrane is mass-produced on the surface 11 of the film carrier 10 fast and continuously. Therefore, the manufacturing cost is reduced due to mass-production of the alginate membrane.

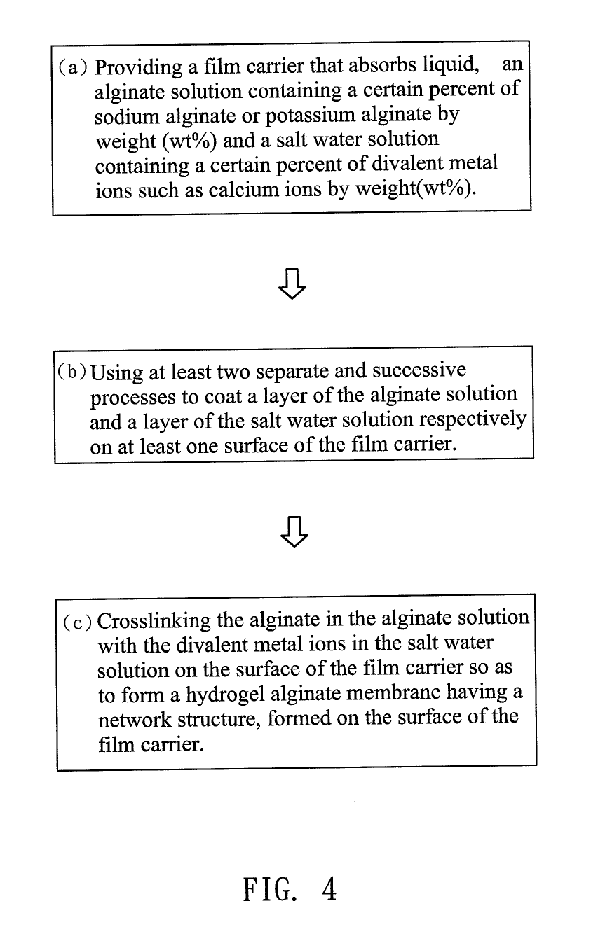

Refer to FIG. 3 and FIG. 4, a manufacturing method of the film 1 containing at least one alginate membrane of the present invention includes following steps:

(a) providing a film carrier 10 that absorbs liquid, an alginate solution 21 containing a certain percent of sodium alginate or potassium alginate by weight (wt %) and a salt water solution 22 containing a certain percent of divalent metal ions such as calcium ions by weight(wt %);

(b) using at least two separate and successive processes to coat a layer of the alginate solution 21 and a layer of the salt water solution 22 respectively on at least one surface 11 of the film carrier 10;

(c) crosslinking the alginate in the alginate solution 21 with the divalent metal ions in the salt water solution 22 on the surface 11 of the film carrier 10 so as to form a hydrogel complex membrane 20 having a network structure, formed on the surface 11 of the film carrier 10, as show in FIG. 1 or filtrating into the surface 11, as shown in FIG. 2.

In the above step (b), the order of coating the alginate solution 21 or the salt water solution 22 on the surface 11 of the film carrier 10 on at least one surface 11 of the film carrier 10 is not limited.

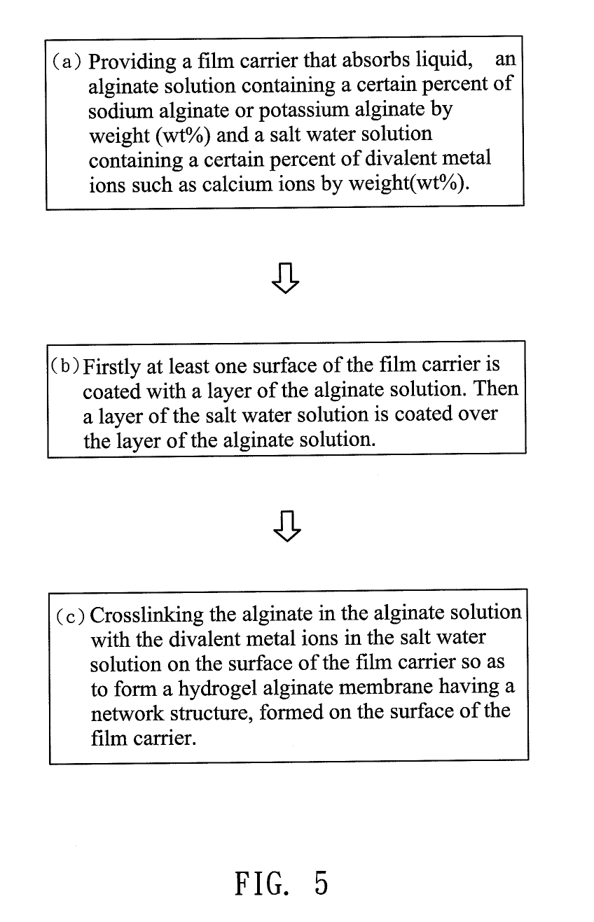

Refer to FIG. 5, in the step (b), firstly at least one surface 11 of the film carrier 10 is coated with a layer of the alginate solution 21. Then a layer of the salt water solution 22 is coated over the layer of the alginate solution 21. While crosslinking the alginate in the alginate solution 21 with the divalent metal ions in the salt water solution 22 on the surface 11 of the film carrier 10, the divalent metal ions in the salt water solution 22 generally infiltrate further into an inner surface of the surface 11 for crosslinking of the alginate. Thus a hydrogel complex membrane 20 with a network structure shown in FIG. 2 is formed above or in the surface 11 of the film carrier 10. In the FIG. 2, the alginate membrane 20 is infiltrated into an infiltration interface 12. The position of the infiltration interface 12 in the surface layer 11 of the film carrier 10 is not limited, depending on the relative concentration of the alginate solution 21 and the salt water solution 22. In this embodiment, the alginate membrane 20 is not separated from the film carrier 10 easily.

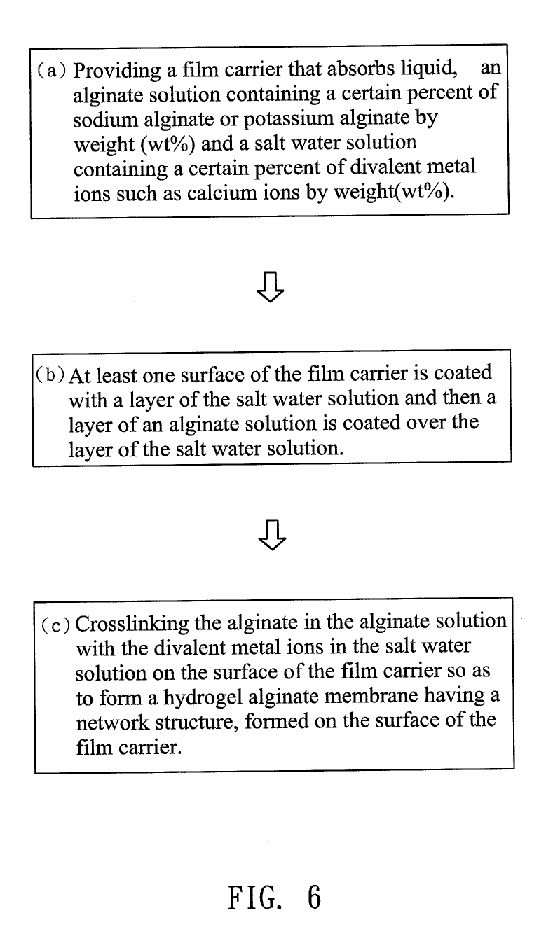

Refer FIG. 6, in the step (b), at least one surface 11 of the film carrier 10 is coated with a layer of the salt water solution 22 and then a layer of an alginate solution 21 is coated over the layer of the salt water solution 22. While crosslinking the alginate in the alginate solution 21 with the divalent metal ions in the salt water solution 22 on the surface 11 of the film carrier 10, the alginate in the alginate solution 21 is firstly crosslinked over the surface 11 of the film carrier 10 to form a hydrogel complex membrane (20) with larger density. Thus the infiltration interface 12 is overlapped with the surface 11, not infiltrating into the inner surface of the surface 11, to crosslink with the divalent metal ions. The hydrogel complex membrane (20) having a network structure is only formed on the upper surface 11 of the film carrier 10, as shown in FIG. 1. The hydrogel complex membrane (20) is the alginate membrane 20 formed on the upper surface 11 of the film carrier 10 in FIG. 1. In this embodiment, the alginate membrane 20 is easy to be separated from the film carrier 10.

Moreover, for convenience of arrangement of coating equipments and coating processes, in the step (b), the first coating is performed by soaking the film carrier 10 into the solution while the second coating is run by spray coating. Take the equipment arrangement and coating procedures shown in FIG. 3 as an example. A roller of film carrier 10 absorbing liquid and having certain length is transported and soaked into a tank filled with an alginate solution 21 by a conveyor. This is the soaking process A shown in the FIG. 3. In this embodiment, the alginate solution 21 is produced by a certain amount of sodium alginate dissolved in water. The weight percent of the sodium alginate in solution ranges from 0.01 to 20 wt % while 1-5 wt % is preferred. In this embodiment, an upper and a lower surfaces (11) of the film carrier 10 are evenly coated with a layer of sodium alginate solution (21). Then the conveyor sends the film carrier 10 already soaked with the sodium alginate solution to be coated with a layer of salt water solution 22 containing a certain weight percent of divalent metal ions by a spray coating process B. In this embodiment, the salt water solution 22 contains divalent calcium ions. The weight percent of the divalent metal ions in the salt water solution ranges from 0.01 to 50 wt % while 1-10 wt % is preferred. The salt water solution 22 is coated over the layer of sodium alginate solution on the surface 11 of the film carrier 10 by spray coating. The salt water solution 22 containing divalent metal ions may further infiltrate into the film carrier 10 and react with the sodium alginate solution in the film carrier 10 for crosslinking, as shown in FIG. 2.

Furthermore, according to the requirement of the thickness of the alginate membrane 20, a plurality sets of spray coating process C is designed. Each set of spray coating process C includes at least one spray coating of the alginate solution 21 C1 and at least one spray coating of the slat water solution C2. After the completion of the spray coating process B, perform the spray coating processes of the alginate solution 21 and the salt water solution in sequence on one surface or two surfaces of the film carrier 10 until the alginate membrane 20 is having the thickness required.

The structure and formation of the film 1 containing at least one alginate membrane 20 of the present invention are revealed in details by following embodiments.

### Embodiment 1

A film carrier 10 is non-woven fabric or cloth made from polypropylene (PP) and is used to absorb 4 wt % calcium lactate that is used as the salt water solution 22. Then 2 wt % sodium alginate solution used as the alginate solution 21 and having the thickness of 1.1 mm is coated over the film carrier 10 with the calcium lactate. After crosslinking, a layer of the alginate membrane 20 with the thickness of about 1 mm is formed on the PP non-woven fabric. The alginate membrane 20 is a hydrogel complex membrane having a network structure. Moreover, when the PP non-woven fabric contacts the salt water solution 22 and the alginate solution 21 by a rough surface of the PP non-woven fabric, the alginate membrane 20 formed is connected with the non-woven fabric (the film carrier 10). When the PP non-woven fabric contacts the salt water solution 22 and the alginate solution 21 by a smooth surface of the PP non-woven fabric, the alginate membrane 20 formed is separated from the non-woven fabric (the film carrier 10).

### Embodiment 2

A film carrier 10 is non-woven fabric or cloth made from polypropylene (PP) and nylon and having the thickness of 0.42 mm. The film carrier 10 absorbing liquid is soaked into 2 wt % sodium alginate solution continuously. After removing extra solution, coat 4 wt % calcium lactate solution on an upper and lower surfaces (11) of the non-woven fabric (the film carrier 10) by spray coating so as to produce a film 1 containing an alginate membrane 20 and having the thickness of about 0.72 mm continuously. This thickness of the film 1 includes the thickness of the fabric. If user wants to produce thicker alginate membrane 20, after the calcium lactate reacting with the sodium alginate, continuously coat one surface or two surfaces of the film 1 produced with a layer of sodium alginate solution and a layer of calcium lactate solution for crosslinking of alginate. Repeat the above procedures until the thickness of the film 1 achieves the required thickness.

### Embodiment 3

A film carrier 10 is non-woven fabric or cloth made from polypropylene (PP) and nylon and having the thickness of 0.42 mm. The film carrier 10 absorbing liquid is soaked into 4 wt % calcium lactate solution continuously. After removing extra solution, coat 2 wt % sodium alginate solution on an upper and lower surfaces (11) of the non-woven fabric (the film carrier 10) by spray coating so as to produce a film 1 containing an alginate membrane 20 and having the thickness of about 0.72 mm continuously. This thickness of the film 1 includes the thickness of the fabric. If user wants to produce thicker alginate membrane 20, after the sodium alginate reacting with the calcium lactate, continuously coat one surface or two surfaces of the film 1 with a layer of calcium lactate solution and a layer of sodium alginate solution for crosslinking of alginate. Repeat the above procedures until the thickness of the film 1 achieves the required thickness.

In addition, while producing the film containing alginate membrane of the present invention, the alginate solution 21 can be added with other materials (additives) such as bioactive components in cosmetics or pigments, vitamins, growth factors, peptides, proteins, minerals, etc. The bioactive components include nutrients in cosmetics, essences in cosmetics, and drug solutions in cosmetics. The pigments are animal/plant extracts.

Compared with the technique available now, a film containing at least one alginate membrane and a manufacturing method of the same have following advantages:

(1) The film can be produced continuously and quickly. This beneficial to mass-production of the alginate membrane and the manufacturing cost is reduced.

(2) The film can be produced by general coating equipments so that the cost of production equipments can be reduced.

(3) After the first crosslinking reaction being completed, at least one surface of the film carrier is spray-coated continuously with a layer of alginate solution and a layer of salt water solution in sequence. Thus a further crosslinking reaction occurs on the surface of the film carrier. The thickness of the alginate membrane is easy to be increased and controlled by at least one further crosslinking reaction. Therefore the performance and applications of the alginate membrane are improved.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A film containing at least one alginate membrane comprising a film carrier that absorbs liquid and at least one alginate membrane; wherein the film carrier is a carrier that absorbs liquid;
the alginate membrane is a hydrogel complex membrane having a network structure formed by crosslinking occurring between at least one layer of alginate solution coated on at least one surface of the film carrier and at least one layer of salt water solution coated on at least one surface of the film carrier; the alginate solution containing a certain percent of alginate such as sodium alginate or potassium alginate by weight while the salt water solution containing a certain percent of divalent metal ions by weight; the layer of the alginate solution and the layer of the salt water solution are coated on the surface of the film carrier by two separate and continuous coating processes; crosslinking between the alginate in the alginate solution and the divalent metal ions in the salt water solution occurring on or into the surface of the film carrier creates the alginate membrane.

2. The film as claimed in claim 1, wherein the film carrier is non-woven fabric, textile, a porous sponge film, or a plastic film.

3. The film as claimed in claim 1, wherein the film carrier is made from synthetic fibers, natural fibers, high molecular polymers or their combinations.

4. The film as claimed in claim 3, wherein the synthetic fibers include polyethylene terephthalate (PET), nylon, acrylics, polypropylene (PP), and poly lactic acid (PLA).

5. The film as claimed in claim 3, wherein the natural fibers include cotton, linen, wool, and silk.

6. The film as claimed in claim 1, wherein the certain percent of sodium alginate or potassium alginate in the alginate solution by weight ranges from 0.01% to 20% by weight.

7. The film as claimed in claim 1, wherein the certain percent of sodium alginate or potassium alginate in the alginate solution by weight ranges from 1% to 5% by weight.

8. The film as claimed in claim 1, wherein the salt water solution containing a certain percent of the divalent metal ions by weight includes calcium lactate solution, calcium chloride solution, calcium gluconate solution, poly-glutamic acid calcium solution, calcium carbonate solution, and calcium sulfate solution.

9. The film as claimed in claim 1, wherein the certain percent of the divalent metal ions by weight ranges from 0.01% to 50% by weight.

10. The film as claimed in claim 1, wherein the certain percent of the divalent metal ions by weight ranges from 1% to 10% by weight.

11. The film as claimed in claim 1, wherein the alginate membrane is integrated with the film carrier when the surface of the film carrier is a rough surface.

12. The film as claimed in claim 1, wherein the alginate membrane is separated from the film carrier when the surface of the film carrier is a smooth surface.

13. The film as claimed in claim 1, wherein the alginate solution is added with additives including nutrients in cosmetics, essences in cosmetics, and drug solutions in cosmetics or pigments.

14. A manufacturing method of a film containing at least one alginate membrane comprising the steps of:
(a) providing a film carrier that absorbs liquid, an alginate solution containing a certain percent of sodium alginate or potassium alginate by weight and a salt water solution containing a certain percent of divalent metal ions by weight;
(b) using at least two separate and successive processes to coat a layer of the alginate solution and a layer of the salt water solution respectively on at least one surface of the film carrier;
(c) crosslinking the alginate in the alginate solution with the divalent metal ions in the salt water solution on the surface of the film carrier so as to produce an alginate membrane that is a hydrogel complex membrane having a network structure, formed on the surface of the film carrier, or formed infiltrating into the surface of the film carrier.

15. The method as claimed in claim 14, wherein the film carrier is non-woven fabric, textile, a porous sponge film, or a plastic film.

16. The method as claimed in claim 14, wherein the film carrier is made from synthetic fibers, natural fibers, high molecular polymers or their combinations.

17. The method as claimed in claim 14, wherein the synthetic fibers include polyethylene terephthalate (PET), nylon, acrylics, polypropylene (PP), and poly lactic acid (PLA).

18. The method as claimed in claim 14, wherein the natural fibers include cotton, linen, wool, and silk.

19. The method as claimed in claim 14, wherein the certain percent of sodium alginate or potassium alginate in the alginate solution by weight ranges from 0.01% to 20% by weight.

20. The method as claimed in claim 14, wherein the certain percent of sodium alginate or potassium alginate in the alginate solution by weight ranges from 1% to 5% by weight.

21. The method as claimed in claim 14, wherein the salt water solution containing a certain percent of the divalent metal ions by weight includes calcium lactate solution, calcium chloride solution, calcium gluconate solution, poly-glutamic acid calcium solution, calcium carbonate solution, and calcium sulfate solution.

22. The method as claimed in claim 14, wherein the certain percent of the divalent metal ions by weight ranges from 0.01% to 50% by weight.

23. The method as claimed in claim 14, wherein the certain percent of the divalent metal ions by weight ranges from 1% to 10% by weight.

24. The method as claimed in claim 14, wherein the alginate membrane is integrated with the film carrier when the surface of the film carrier is a rough surface.

25. The method as claimed in claim 14, wherein alginate membrane is separated from the film carrier when the surface of the film carrier is a smooth surface.

26. The method as claimed in claim 14, wherein in the step (b), a layer of the alginate solution is firstly coated on the at least one surface of the film carrier and then a layer of the salt water solution is coated over the layer of the alginate solution.

27. The method as claimed in claim 14, wherein in the step (b), a layer of the water salt solution is firstly coated on the at least one surface of the film carrier and then a layer of the alginate solution is coated over the layer of the alginate solution.

28. The method as claimed in claim 14, wherein in the step (b), a previous coating is performed by soaking and the next coating is performed by spray coating.

29. The method as claimed in claim 14, wherein after the crosslinking in the step (c) being completed, the method further includes a step of performing a spray coating process of the alginate solution and a spray coating process of the salt water solution in order on the surface of the film carrier for crosslinking and increasing thickness of the alginate membrane.

30. The method as claimed in claim 14, wherein alginate solution is added with additives including nutrients in cosmetics, essences in cosmetics, and drug solutions in cosmetics or pigments.
